(19) United States
(12) Patent Application Publication
(10) Pub. No.: US 2026/0137366 A1
(43) Pub. Date: May 21, 2026

(54) CONTACT DETERMINATION METHOD ON BASIS OF MEDICAL IMAGE, AND COMPUTER PROGRAM PERFORMING SAME

(71) Applicant: AIRS MEDICAL INC., Seoul (KR)

(72) Inventor: Youngsik KIM, Seoul (KR)

(21) Appl. No.: 19/114,469

(22) PCT Filed: Jun. 21, 2023
(86) PCT No.: PCT/KR2023/008620

§ 371 Date: Mar. 24, 2025

(30) Foreign Application Priority Data
Oct. 14, 2022 (KR) 10-2022-0132185

(51) Int. Cl. A61B 8/00; G06V 10/26; G06V 10/74; G06V 20/50

(52) U.S. Cl. CPC A61B 8/4245; G06V 10/26; G06V 10/761; G06V 20/50; G06V 2201/03

(57) ABSTRACT

A method of determining contact between an ultrasound probe and a body part according to one embodiment of the present disclosure, the method being performed by a computing device, the method including: acquiring a plurality of reference images via the ultrasound probe; generating a contact determination criterion for the determination of whether there is contact between the ultrasound probe and the body part and a valid pressure range for contact pressure based on the plurality of reference images; acquiring a candidate image via the ultrasound probe; determining whether the candidate image satisfies the contact determination criterion and the valid pressure range; and, when the candidate image satisfies the contact determination criterion and the valid pressure range, determining the candidate image to be a valid image acquired in a state where the contact pressure between the ultrasound probe and the body part is appropriate.

### TECHNICAL FIELD

The present disclosure relates to a contact determination method based on a medical image and a computer program performing the same, and more particularly, to a contact determination method for determining whether there is contact between an ultrasound probe and a body part by using an ultrasonic image, and a computer program performing the same.

### BACKGROUND ART

Medical images are data that allows users to understand the physical states of various organs in the human body for the diagnosis and treatment of diseases. As medical imaging devices continue to be developed, medical images are becoming more diverse, as in the case of ultrasonic images, X-ray images, computed tomography (CT) images, positron emission tomography (PET) images, or magnetic resonance imaging (MRI) images.

Among such devices, an ultrasound imaging device acquires images of parts inside the body by radiating the ultrasonic signals, generated from the transducer of a probe, onto the body and receiving information about the ultrasonic echo signals reflected from the body.

An ultrasound imaging device, by its nature, radiates ultrasonic signals onto the body while an ultrasound probe and a body surface are in contact. Accordingly, the pressure applied by a user who manipulates the ultrasound probe may be applied to the body via the ultrasound probe. Since contact needs to occur between the ultrasound probe and the body surface, the user needs to apply pressure. However, when the pressure is excessive, the body may be deformed and an ultrasonic image may be distorted.

Medical procedures such as diagnosis need to be performed using ultrasonic images acquired under an appropriate pressure between an ultrasound probe and a body surface. Therefore, there is a demand for a technology for determining whether the pressure between the ultrasound probe and the body surface is appropriate.

### DISCLOSURE

### Technical Problem

The present disclosure is intended to overcome the problems of the above-described conventional art, and is directed to a medical image-based contact determination method for determining whether there is contact between an ultrasound probe and a body part and whether the contact pressure is appropriate by using an ultrasonic image, and a computer program performing the same.

However, the technical problems to be overcome by the present embodiment are not limited to the technical problem described above, and other technical problems may be present.

### Technical Solution

According to one embodiment of the present disclosure for achieving the above-described object, there is disclosed a method of determining contact between an ultrasound probe and a body part that is performed by a computing device. The method includes: acquiring a plurality of reference images via the ultrasound probe; generating a contact determination criterion for the determination of whether there is contact between the ultrasound probe and the body part and a valid pressure range for contact pressure based on the plurality of reference images; acquiring a candidate image via the ultrasound probe; determining whether the candidate image satisfies the contact determination criterion and the valid pressure range; and, when the candidate image satisfies the contact determination criterion and the valid pressure range, determining the candidate image to be a valid image acquired in a state where the contact pressure between the ultrasound probe and the body part is appropriate.

Alternatively, the generating includes: generating the contact determination criterion so that, when the similarity between the plurality of reference images and the candidate image exceeds the contact determination criterion, it is determined that contact has occurred between the ultrasound probe and the body part.

Alternatively, the generating the contact determination criterion includes: calculating similarities between the plurality of reference images, and generating the contact determination criterion based on the smallest value of the calculated similarities.

Alternatively, the generating the contact determination criterion includes: dividing each of the plurality of reference images into patches; and generating the contact determination criterion based on the smallest value of the similarities between patches generated from the plurality of reference images.

Alternatively, the determining whether the candidate image satisfies the contact determination criterion and the valid pressure range includes: when the similarity between a patch of the plurality of reference images and a patch of the candidate image satisfies the contact determination criterion, determining the patch of the candidate image to be a contact occurrence patch.

Alternatively, the determining the candidate image to be a valid image includes: when the number of contact occurrence patches included in the candidate image exceeds a first reference number included in the valid pressure range, determining the candidate image to be the valid image.

Alternatively, the determining the candidate image to be a valid image includes: when the number of consecutively arranged contact occurrence patches included in the candidate image exceeds a second reference number included in the valid pressure range, determining the candidate image to be the valid image.

Alternatively, the method further includes: determining the time when the valid image is acquired to be the time when the contact pressure between the ultrasound probe and the body part is in an appropriate state.

According to one embodiment of the present disclosure for achieving the above-described object, there is disclosed a method of determining contact between an ultrasound probe and a body part that is performed by a computing device. The method includes: acquiring a candidate image via the ultrasound probe; and determining whether the candidate image is a valid image acquired in a state where the contact pressure between the ultrasound probe and the body part is appropriate by using a first artificial intelligence model trained to determine whether there is contact between the ultrasound probe and the body part or a second artificial intelligence model trained to infer the contact pressure between the ultrasound probe and the body part.

Alternatively, the determining includes: dividing the candidate image into patches; determining whether each of the patches is a contact occurrence patch by using the first artificial intelligence model; and determining the candidate image to be the valid image based on the number of contact occurrence patches included in the candidate image.

Alternatively, the determining includes: when the number of contact occurrence patches included in the candidate image exceeds a first reference number, determining the candidate image to be the valid image.

Alternatively, the determining includes: when the number of consecutively arranged contact occurrence patches included in the candidate image exceeds a second reference number, determining the candidate image to be the valid image.

According to one embodiment of the present disclosure for achieving the above-described object, there is disclosed a computer program stored in a computer-readable storage medium. When executed on at least one processor, the computer program causes the processor to perform operations for determining contact between an ultrasound probe and a body part. In this case, the operations include: acquiring a plurality of reference images via the ultrasound probe; generating a contact determination criterion for the determination of whether there is contact between the ultrasound probe and the body part and a valid pressure range for contact pressure based on the plurality of reference images; acquiring a candidate image via the ultrasound probe; determining whether the candidate image satisfies the contact determination criterion and the valid pressure range; and, when the candidate image satisfies the contact determination criterion and the valid pressure range, determining the candidate image to be a valid image acquired in a state where the contact pressure between the ultrasound probe and the body part is appropriate.

### Advantageous Effects

According to one embodiment of the present disclosure, there is no need to add a separate device for measuring pressure to the hardware that acquires ultrasonic images, so that installation and usage are easy, and the contact pressure may be determined using only an ultrasonic image, so that a high computation speed can be provided.

Furthermore, according to one embodiment of the present disclosure, the criteria are set using ultrasonic images acquired from the device rather than using a uniform contact determination criterion and a uniform valid pressure range, so that the accuracy of contact pressure determination can be increased regardless of changes in environmental factors.

### DESCRIPTION OF DRAWINGS

FIG. 1 is a block diagram of a computing device according to one embodiment of the present disclosure;

FIG. 2 is a block diagram of a contact pressure determination device according to one embodiment of the present disclosure;

FIG. 3 is an exemplary diagram showing reference images according to one embodiment of the present disclosure;

FIG. 4 is a flowchart showing a method of operating a contact pressure determination device for determining a valid image based on a rule base according to one embodiment of the present disclosure;

FIG. 5 is a flowchart showing a method of operating a contact pressure determination device for determining a valid image based on a rule base according to one embodiment of the present disclosure; and

FIG. 6 is a flowchart showing a method of operating a contact pressure determination device for determining a valid image based on artificial intelligence according to one embodiment of the present disclosure.

### MODE FOR INVENTION

Embodiments of the present disclosure will be described in detail below with reference to the accompanying drawings so that those having ordinary skill in the art of the present disclosure (hereinafter referred to as those skilled in the art) can easily implement the present disclosure. The embodiments presented in the present disclosure are provided to enable those skilled in the art to use or practice the content of the present disclosure. Accordingly, various modifications to embodiments of the present disclosure will be apparent to those skilled in the art. That is, the present disclosure may be implemented in various different forms and is not limited to the following embodiments.

The same or similar reference numerals denote the same or similar components throughout the specification of the present disclosure. Additionally, in order to clearly describe the present disclosure, reference numerals for parts that are not related to the description of the present disclosure may be omitted in the drawings.

The term “or” used herein is intended not to mean an exclusive “or” but to mean an inclusive “or.” That is, unless otherwise specified herein or the meaning is not clear from the context, the clause “X uses A or B” should be understood to mean one of the natural inclusive substitutions. For example, unless otherwise specified herein or the meaning is not clear from the context, the clause “X uses A or B” may be interpreted as any one of a case where X uses A, a case where X uses B, and a case where X uses both A and B.

The term “and/or” used herein should be understood to refer to and include all possible combinations of one or more of listed related concepts.

The terms “include” and/or “including” used herein should be understood to mean that specific features and/or components are present. However, the terms “include” and/or “including” should be understood as not excluding the presence or addition of one or more other features, one or more other components, and/or combinations thereof.

Unless otherwise specified herein or unless the context clearly indicates a singular form, the singular form should generally be construed to include “one or more.”

The term “N-th (N is a natural number)” used herein may be understood as an expression used to distinguish the components of the present disclosure according to a predetermined criterion such as a functional perspective, a structural perspective, or the convenience of description. For example, in the present disclosure, components performing different functional roles may be distinguished as a first component or a second component. However, components that are substantially the same within the technical spirit of the present disclosure but should be distinguished for the convenience of description may also be distinguished as a first component or a second component.

Meanwhile, the term “module” or “unit” used herein may be understood as a term referring to an independent functional unit processing computing resources, such as a computer-related entity, firmware, software or part thereof, hardware or part thereof, or a combination of software and hardware. In this case, the “module” or “unit” may be a unit composed of a single component, or may be a unit expressed as a combination or set of multiple components. For example, in the narrow sense, the term “module” or “unit” may refer to a hardware component or set of components of a computing device, an application program performing a specific function of software, a procedure implemented through the execution of software, a set of instructions for the execution of a program, or the like. Additionally, in the broad sense, the term “module” or “unit” may refer to a computing device itself constituting part of a system, an application running on the computing device, or the like. However, the above-described concepts are only examples, and the concept of “module” or “unit” may be defined in various manners within a range understandable to those skilled in the art based on the content of the present disclosure.

The term “model” used herein may be understood as a system implemented using mathematical concepts and language to solve a specific problem, a set of software units intended to solve a specific problem, or an abstract model for a process intended to solve a specific problem. For example, a neural network “model” may refer to an overall system implemented as a neural network that is provided with problem-solving capabilities through training. In this case, the neural network may be provided with problem-solving capabilities by optimizing parameters connecting nodes or neurons through training. The neural network “model” may include a single neural network, or a neural network set in which multiple neural networks are combined together.

The term “image” used herein may refer to multi-dimensional data composed of discrete image elements (e.g., pixels in a two-dimensional image and voxels in a three-dimensional image). For example, the image may include an medical image acquired by a medical imaging device such as an ultrasound imaging device, an infrared imaging device, a magnetic resonance imaging device, a computed tomography (CT) device, or an X-ray imaging device.

The foregoing descriptions of the terms are intended to help to understand the present disclosure. Accordingly, it should be noted that unless the above-described terms are explicitly described as limiting the content of the present disclosure, they are not used in the sense of limiting the technical spirit of the present disclosure.

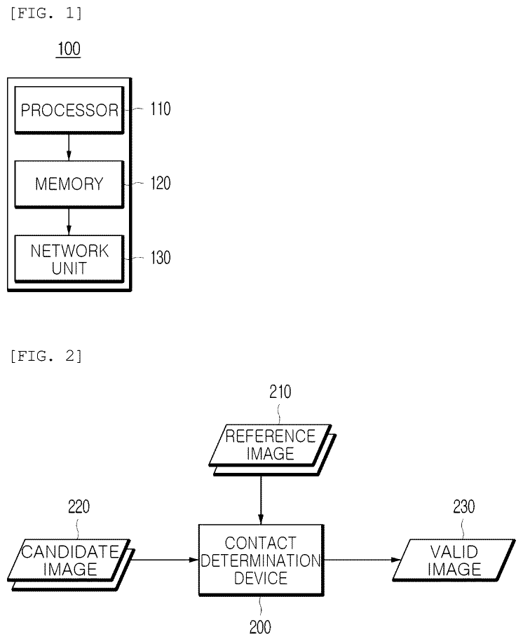

FIG. 1 is a block diagram of a computing device according to one embodiment of the present disclosure.

A computing device 100 according to one embodiment of the present disclosure may be a hardware device or a part of a hardware device that performs the comprehensive processing and computation of data, or may be a software-based computing environment connected to a communication network. For example, the computing device 100 may be a server, which is a main agent that performs intensive data processing functions and shares resources, or may be a client that shares resources through interaction with a server. Furthermore, the computing device 100 may be a cloud system that allows pluralities of servers and clients to comprehensively process data while interacting with each other. Additionally, the computing device 100 may be a medical robot that supports or assists with overall medical procedures performed in a medical field. In this case, the medical robot may include a venipuncture robot that includes a blood collection or intravenous injection (IV) function for diagnosing diseases, transfusions, etc. The above description is only one example related to the type of computing device 100, so that the type of computing device 100 can be configured in various manners within a range understandable to those skilled in the art based on the contents of the present disclosure. The above description is only one example related to the type of computing device 100, so that the type of computing device 100 can be configured in various manners within a range understandable to those skilled in the art based on the contents of the present disclosure.

Referring to FIG. 1, the computing device 100 according to one embodiment of the present disclosure may include a processor 110, memory 120, and a network unit 130. However, FIG. 1 shows only an example, and the computing device 100 may include other components for implementing a computing environment. Alternatively, only some of the components disclosed above may be included in the computing device 100.

The processor 110 according to one embodiment of the present disclosure may be understood as a constituent unit including hardware and/or software for performing computing operation. For example, the processor 110 may read a computer program and perform data processing for machine learning. The processor 110 may process operation processes such as the processing of input data for machine learning, the extraction of features for machine learning, and the computation of errors based on backpropagation. The processor 110 for performing such data processing may include a central processing unit (CPU), a general purpose graphics processing unit (GPGPU), a tensor processing unit (TPU), an application specific integrated circuit (ASIC), or a field programmable gate array (FPGA). Since the types of processor 110 described above are only examples, the type of processor 110 may be configured in various manners within a range understandable to those skilled in the art based on the content of the present disclosure.

According to one embodiment of the present disclosure, in the process of acquiring an ultrasonic image inside the body via an ultrasound probe, the processor 110 may determine whether the ultrasound probe is in contact with a body part and whether the contact pressure applied by the ultrasound probe to the body part is appropriate. When an ultrasonic image is acquired, the processor 110 may determine whether the ultrasound probe is in contact with the body part and the contact pressure between the ultrasound probe and the body part by using the corresponding ultrasonic image acquired via the ultrasound probe.

The processor 110 may generate a contact determination criterion for the determination of whether the ultrasound probe and the body part are in contact with each other.

In order to observe elements such as blood vessels inside the body via an ultrasonic image, the ultrasound probe needs to make appropriate contact with the body part. That is, when the pressure applied by the ultrasound probe is excessively small, the ultrasound probe may not make sufficient contact with a body part due to the curvature of the body part, making it difficult to acquire information about a desired part. In contrast, when the pressure applied by the ultrasound probe is excessively large, the shapes of the elements inside the body may be deformed due to the pressure of the ultrasound probe. Accordingly, the processor 110 may generate a valid pressure range that serves as a criterion for the determination of whether the contact pressure between the ultrasound probe and the body part is appropriate.

As one embodiment, the processor 110 may receive a plurality of ultrasonic images acquired while the ultrasound probe is stationary. The processor 110 may generate a contact determination criterion and a valid pressure range based on the similarity between the acquired ultrasonic images by comparing the acquired ultrasonic images with each other. The processor 110 may generate a contact determination criterion by dividing each of the ultrasonic images into a plurality of patches and calculating similarities on a per-patch basis. The processor 110 may receive an ultrasonic image acquired while the ultrasound probe moves toward the body part, and may determine whether the ultrasonic image is a valid image acquired in an appropriate pressure state based on the contact determination criterion and the valid pressure range.

As one embodiment, the processor 110 may train a first artificial intelligence model by using an ultrasonic image, acquired in a state where contact has occurred between the ultrasound probe and the body part, as a training image. Furthermore, the processor 110 may train a second artificial intelligence model by using an image, acquired in a state where the contact pressure between the ultrasound probe and the body part is within the valid pressure range, as a training image. Furthermore, the second artificial intelligence model may be trained to output the contact pressure between the ultrasound probe and the body part as a numerical value. The first artificial intelligence model and the second artificial intelligence model may include at least one neural network. The neural network may include network models such as a deep neural network (DNN), a recurrent neural network (RNN), a bidirectional recurrent deep neural network (BRDNN), a multilayer perceptron (MLP), and a convolutional neural network (CNN), but is not limited thereto. The processor 110 may receive an ultrasonic image acquired while the ultrasound probe moves toward the body part, and may determine whether the ultrasonic image is a valid image by inputting the ultrasonic image to the first artificial intelligence model or second artificial intelligence model.

The processor 110 may determine the time when a valid image is acquired to be the time when the ultrasound probe comes into appropriate contact with the body part. The processor 110 may provide a notification to a venipuncture device at the time when the valid image is acquired. Accordingly, based on the valid image, the venipuncture device may perform an operation of identifying blood vessel candidates that are targets for invasion inside the body, an operation of determining a blood vessel target for invasion among the blood vessel candidates, and an operation of performing an invasion on the blood vessel target for invasion.

According to one embodiment of the present disclosure, there is no need to add a separate device for measuring pressure to the hardware that acquires ultrasonic images, so that installation and usage are easy, and the contact pressure may be determined using only an ultrasonic image, so that a high computation speed can be provided. Furthermore, rather than using a uniform contact determination criterion and a uniform valid pressure range, the criteria are set using ultrasonic images acquired when the device is operated. Accordingly, the accuracy of determination of the contact pressure may be increased by adaptively setting the contact determination criterion and the valid pressure range in response to changes in environmental factors.

When necessary, the processor 110 may generate a user interface that provides an environment for interaction with a user of the computing device 100 or a user of any client. The processor 110 may generate a user interface that enables functions such as the output, modification, change, and addition of data to be implemented based on external input signals received from a user. Since the roles of the user interface described above are only examples, the role of the user interface may be defined in various manners within a range understandable to those skilled in the art based on the content of the present disclosure.

The memory 120 according to one embodiment of the present disclosure may be understood as a constituent unit including hardware and/or software for storing and managing data that is processed in the computing device 100. That is, the memory 120 may store any type of data generated or determined by the processor 110 and any type of data received by the network unit 130. For example, the memory 120 may include at least one type of storage medium of a flash memory type, hard disk type, multimedia card micro type, and card type memory, random access memory (RAM), static random access memory (SRAM), read-only memory (ROM), electrically erasable programmable read-only memory (EEPROM), programmable read-only memory (PROM), magnetic memory, a magnetic disk, and an optical disk. Furthermore, the memory 120 may include a database system that controls and manages data in a predetermined system. Since the types of memory 120 described above are only examples, the type of memory 120 may be configured in various manners within a range understandable to those skilled in the art based on the content of the present disclosure.

The memory 120 may structure, organize and manage data required for the processor 110 to perform operations, combinations of data, and program codes executable by the processor 110.

The memory 120 may store the ultrasonic images received via the network unit 130, which will be described later. The memory 120 may store the program codes that enable operation to generate the contact determination criterion and the valid pressure range based on ultrasonic images and the contact determination criterion and valid pressure range that are generated as the program codes are executed.

The network unit 130 according to one embodiment of the present disclosure may be understood as a constituent unit that transmits and receives data through any type of known wired/wireless communication system. For example, the network unit 130 may perform data transmission and reception using a wired/wireless communication system such as a local area network (LAN), a wideband code division multiple access (WCDMA) network, a long term evolution (LTE) network, the wireless broadband Internet (WiBro), a 5th generation mobile communication (5G) network, an ultra-wideband wireless communication network, a ZigBee network, a radio frequency (RF) communication network, a wireless LAN, a wireless fidelity network, a near field communication (NFC) network, or a Bluetooth network. Since the above-described communication systems are only examples, the wired/wireless communication system for the data transmission and reception of the network unit 130 may be applied in various manners other than the above-described examples.

The network unit 130 may receive the data, required for the processor 110 to perform operations, through wired or wireless communication with any system, any client, or the like. Furthermore, the network unit 130 may transmit the data, generated through the operation of the processor 110, through wired or wireless communication with any system, any client, or the like. For example, the network unit 130 may receive ultrasonic images through communication with a picture archiving and communication system, a cloud server that performs tasks such as the standardization of medical data, or a medical robot or through communication with a medical image acquisition device.

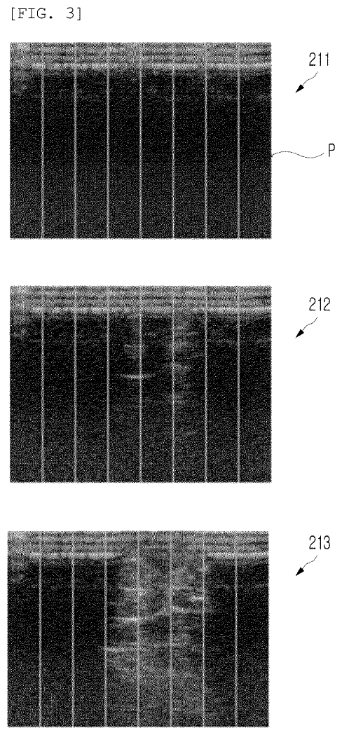

FIG. 2 is a block diagram of a contact pressure determination device according to one embodiment of the present disclosure, and FIG. 3 is an exemplary diagram showing reference images according to one embodiment of the present disclosure.

Referring to FIGS. 2 and 3 together, a contact determination device 200 may be one embodiment of the computing device 100 of FIG. 1, and may be a component included in a venipuncture device, e.g., a blood collection device.

The contact pressure determination device may acquire reference images 210 generated from an ultrasound probe, and may generate a contact determination criterion for the determination of whether the ultrasound probe and a body part are in contact and a valid pressure range for the determination of whether the contact pressure applied by the ultrasound probe to the body part is appropriate.

The reference images 210 may include a plurality of ultrasonic images, e.g., an initial image 211, a first reference image 212, and a second reference image 213. The initial image 211 may be taken after distilled water has been sprayed onto the ultrasound probe, and the first and second reference images 212 and 213 may be taken after the distilled water has been sprayed but before the ultrasound probe is moved. That is, the reference images 210 may collectively refer to a plurality of ultrasonic images acquired before the ultrasound probe starts a vertical downward movement. Although the three reference images 210 are described as an example in FIG. 3, the number of reference images 210 required to generate a contact determination criterion is not limited thereto.

The contact determination device 200 may generate a contact determination criterion based on the similarity between the images. The contact determination device 200 may divide each of the images into predetermined regions and calculate the similarities between corresponding regions. For example, the contact determination device 200 may divide each of the images into patches P and calculate the similarities between corresponding patches P.

Referring to FIG. 3, the initial image 211, the first reference image 212, and the second reference image 213 may each be divided into eight patches. The contact determination device 200 may calculate the similarities between the patches of the initial image 211 and the patches of the first reference image 212, and may calculate the similarities between the patches of the initial image 211 and the patches of the second reference image 213. The contact determination device 200 may calculate the similarities between patches present at corresponding locations. Accordingly, 16 similarity values may be calculated. The contact determination device 200 may generate the smallest similarity value as the contact determination criterion.

Meanwhile, a method for calculating similarities between images is not limited to a specific one, and various methods such as the structural similarity index map (SSIM), the mean square error (MSE), and image hashes (an average hash, a perceptive hash, and a difference hash) may be used.

The contact determination device 200 may determine a region satisfying the contact determination criterion to be a contact occurrence region. More specifically, the contact determination device 200 may determine a patch satisfying the contact determination criterion to be a contact occurrence patch.

The contact determination device 200 may generate the valid pressure range based on the contact determination criterion. The contact determination device 200 may generate the valid pressure range based on the number or locations of contact occurrence patches. For example, when any ultrasonic image includes a first reference number of contact occurrence patches, the contact determination device 200 may determine the ultrasonic image to be a valid image 230. That is, the contact determination device 200 may set the first reference number as the valid pressure range. For example, when a second reference number of contact occurrence patches included in any ultrasonic image are arranged in succession, the contact determination device 200 may determine the ultrasonic image to be the valid image 230. That is, the contact determination device 200 may set the second reference number as the valid pressure range. The valid pressure range, i.e., the first reference number and the second reference number, may be determined and changed depending on the characteristics of the body part, the characteristics of the blood vessels included in the ultrasonic image, the size and resolution of the ultrasonic image, the size of the patches, and/or the like.

The contact determination device 200 may determine whether the ultrasound probe and the body part are in contact with each other based on the contact determination criteria by using candidate images 220 acquired from the ultrasound probe. The contact determination device 200 may determine whether the ultrasound probe and the body part are in contact at an appropriate contact pressure based on the valid pressure range. In this time, the reference images 210 may be acquired while the ultrasound probe is stationary, and the candidate images 220 may be acquired while the ultrasound probe is moving.

The contact determination device 200 may calculate the similarity between the candidate image 220 and the reference image 210 (e.g., the initial image), and may determine that contact has occurred between the ultrasound probe and the body part when the similarity exceeds the contact determination criterion. The contact determination device 200 may divide the candidate image 220 into predetermined regions, e.g., patches P, and may calculate the similarities for the respective patches P. That is, the contact determination device 200 may calculate the similarities between the patches of the candidate image 220 and the patches of the initial image, and may determine whether the number of patches exceeding the contact determination criterion (i.e., the number of patches where contact has occurred) exceeds the contact determination criterion.

The contact determination device 200 may determine the candidate image 220 to be the valid image 230 when the candidate image 220 satisfies the valid pressure range. For example, the contact determination device 200 may determine whether the number of contact occurrence patches in the candidate image 220 exceeds the first reference number, and may determine the candidate image 220 to be the valid image 230 when the number of contact occurrence patches exceeds the first reference number. Alternatively, the contact determination device 200 may determine whether the number of contact occurrence patches consecutively arranged in the candidate image 220 exceeds the second reference number. When the number of consecutively arranged contact occurrence patches exceeds the second reference number, the contact determination device 200 may then determine the candidate image 220 to be the valid image 230.

The contact determination device 200 may determine the time when the valid image 230 is acquired or the time when the valid image 230 is generated to be the time when the ultrasound probe and the body part come into contact at an appropriate contact pressure.

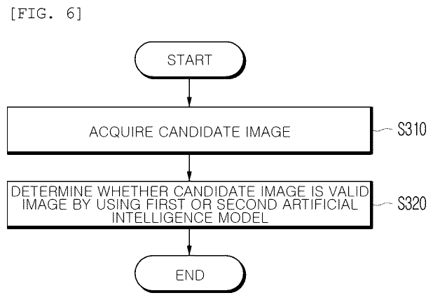

The contact determination device 200 may determine the candidate image 220 to be the valid image 230 by using an artificial intelligence model. In this case, training data for the training of the artificial intelligence model may be used. This will be described in detail later with reference to FIG. 6.

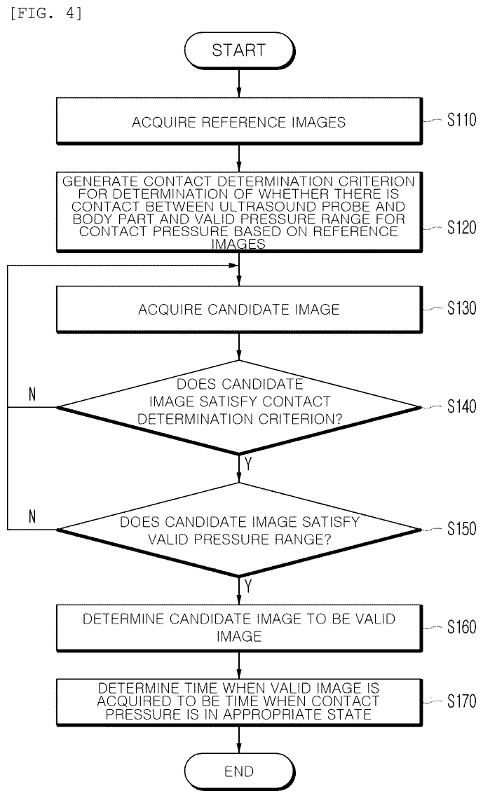

FIG. 4 is a flowchart showing a method of operating a contact pressure determination device for determining a valid image based on a rule base according to one embodiment of the present disclosure.

Referring to FIG. 4, the contact determination device may be one embodiment of the computing device 100 of FIG. 1.

The contact determination device may acquire reference images in step S110. The contact determination device may acquire the reference images obtained by imaging a body part while the ultrasound probe is stationary via the ultrasound probe. The reference images may be a plurality of reference images.

Based on the reference images, the contact determination device may generate a contact determination criterion for the determination of whether contact has occurred between the ultrasound probe and the body part and a valid pressure range for the contact pressure in step S120.

The contact determination criterion may include a similarity value between images. The contact determination device may generate a contact determination criterion to determine that contact has occurred between the ultrasound probe and the body part when the similarity between the reference image and the candidate image to be acquired exceeds the contact determination criterion. When the reference images include a plurality of ultrasonic images, the contact determination device may calculate the similarities between the reference images and generate the contact determination criterion based on the smallest value of the similarities.

The valid pressure range may be generated based on a region satisfying the contact determination criterion in the ultrasonic image. The region satisfying the contact determination criterion in the ultrasonic image may be referred to as a contact occurrence region, and the contact occurrence region may be referred to as a contact occurrence patch when the region is a patch. The valid pressure range may be generated based on the number of contact occurrence patches in the ultrasonic image.

The contact determination device may acquire a candidate image in step S130. The contact determination device may acquire the reference images and then acquire a candidate image after a specific time.

The contact determination device may determine whether the candidate image satisfies the contact determination criterion in step S140. The contact determination device may calculate the similarity between each of the reference images and the candidate image, and may determine that contact has occurred between the ultrasound probe and the body part when the similarity exceeds the contact determination criterion.

When the candidate image satisfies the contact determination criterion, the contact determination device may determine whether the candidate image satisfies the valid pressure range in step S150. The contact determination device may identify contact occurrence regions in the candidate image and determine whether the regions satisfy the valid pressure range. The contact determination device may compare the size and arrangement of the contact determination regions in the candidate image with the valid pressure range.

When the candidate image satisfies the valid pressure range, the contact determination device may determine the candidate image to be a valid image in step S160.

The contact determination device may determine the time when a valid image is acquired to be the time when the contact pressure is in an appropriate state in step S170. The contact determination device may provide a notification to an external component connected to 200, e.g., a venipuncture device, at the time when the valid image is acquired.

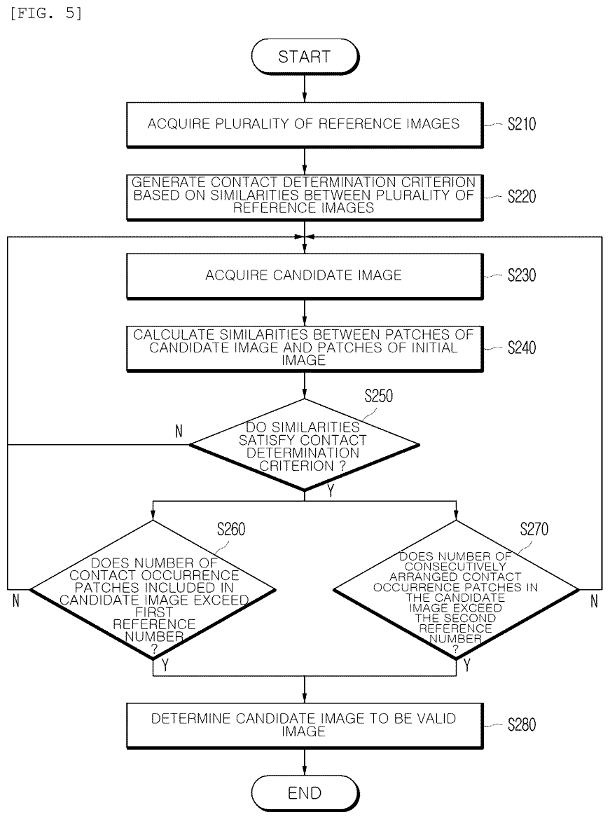

FIG. 5 is a flowchart showing a method of operating a contact pressure determination device for determining a valid image based on a rule base according to one embodiment of the present disclosure.

Referring to FIG. 5, the contact determination device may be one embodiment of the computing device 100 of FIG. 1. The contact determination device may divide an ultrasonic image into patches and then perform a contact pressure determination operation. That is, the contact determination device may perform an operation of dividing an acquired ultrasonic image into patches. The size and number of patches may vary depending on the ultrasonic image.

The contact determination device may acquire a plurality of reference images in step S210. The contact determination device may acquire ultrasonic images before the ultrasound probe starts its downward movement, and may use them as the reference images. For example, the reference images may include an initial image taken after distilled water has been sprayed onto the ultrasound probe and first and second reference images taken after the distilled water has been sprayed but before the ultrasound probe is moved.

The contact determination device may generate a contact determination criterion based on the similarities between the plurality of reference images in step S220. The contact determination device may calculate the similarities between the plurality of reference images, and may generate the contact determination criterion based on the smallest value of the calculated similarities. The contact determination device may divide each of the plurality of reference images into patches, and may generate the contact determination criterion based on the smallest value of the similarities between patches.

For example, the contact determination device may divide each of the initial image, the first reference image, and the second reference image into patches. The contact determination device may calculate the similarities between the patches of the initial image and the patches of the first reference image, and may calculate the similarities between the patches of the initial image and the patches of the second reference image. The contact determination device may calculate the similarities between patches at corresponding locations in the images. The contact determination device may generate a contact determination criterion based on the smallest value of the similarity values calculated for the respective patches.

The contact determination device may generate a valid pressure range based on the number of contact occurrence patches that satisfy the contact determination criterion. The valid pressure range may include a first reference number and/or a second reference number. For example, the contact determination device may determine that a contact pressure falls within the valid pressure range when the number of contact occurrence patches exceeds the first reference number. For example, the contact determination device may determine that a contact pressure falls within the valid pressure range when the number of consecutively arranged contact occurrence patches exceeds the second reference number.

The contact determination device may acquire a candidate image taken while the ultrasound probe moves in step S230.

The contact determination device may calculate the similarities between the patches of the candidate image and the patches of the initial image in step S240. The contact determination device may calculate the similarities between patches at corresponding locations of the candidate image and the initial image.

The contact determination device may determine whether each of the similarities satisfies the contact determination criterion in step S250. The contact determination device may determine a patch satisfying the contact determination criterion to be a contact occurrence patch.

The contact determination device may determine whether the number of contact occurrence patches in the candidate image exceeds the first reference number in step S260. The contact determination device may determine whether the number of consecutively arranged contact occurrence patches in the candidate image exceeds the second reference number in step S270. Steps S260 and S270 may be performed selectively or in parallel. Alternatively, they may be performed sequentially.

When the number of contact occurrence patches exceeds the first reference number in step S260 or the number of consecutively arranged contact occurrence patches exceeds the second reference number in step S270, the contact determination device may determine the candidate image to be a valid image in step S280. Accordingly, the contact determination device may determine the time when the valid image is acquired to be the time when the contact pressure between the ultrasound probe and the body part is in an appropriate state.

In the case where the similarity does not satisfy the contact determination criterion in step S250, in the case where the number of contact occurrence patches is smaller than or equal to the first reference number in step S260, or in the case where the number of consecutively arranged contact occurrence patches does not exceed the second reference number and the contact occurrence patches are not arranged in succession in step S270, the contact determination device may return to step S230 and re-acquire a candidate image.

FIG. 6 is a flowchart showing a method of operating a contact pressure determination device for determining a valid image based on artificial intelligence according to one embodiment of the present disclosure.

Referring to FIG. 6, the contact determination device may be one embodiment of the computing device 100 of FIG. 1. Before step S320, a first artificial intelligence model and a second artificial intelligence model may be trained.

The first artificial intelligence model may be trained to determine whether contact has occurred between the ultrasound probe and a body part.

The second artificial intelligence model may be trained to determine whether the ultrasonic image is a valid image acquired within the valid pressure range in order to determine whether the contact pressure between the ultrasound probe and the body part falls within the valid pressure range.

The first artificial intelligence model may use an ultrasonic image, acquired in a state where contact has occurred between the ultrasound probe and the body part, as a training image. The second artificial intelligence model may use an ultrasonic image, acquired in a state where the contact pressure between the ultrasound probe and the body part satisfies the valid pressure range, as a training image. The second artificial intelligence model may be trained, with an ultrasonic image acquired in a state where the contact pressure is lower than the valid pressure range being labeled as 0, an ultrasonic image acquired in a state where the contact pressure falls within the valid pressure range being labeled as 1, and an ultrasonic image acquired in a state where the contact pressure exceeds the valid pressure range being labeled as 2.

Alternatively, the second artificial intelligence model may be trained to output the contact pressure between the ultrasound probe and the body part as a numerical value. In this case, the second artificial intelligence model may use a plurality of ultrasonic images based on the contact pressures as training images.

The first and second ultrasonic image models may be trained based on portions, e.g., patches, of ultrasonic images.

The contact determination device may acquire a candidate image in step S310. The candidate image may be an ultrasonic image acquired after the ultrasound probe has started a downward movement.

The contact determination device may determine whether the candidate image is a valid image acquired in a state of appropriate contact pressure between the ultrasound probe and the body part by using the trained first or second artificial intelligence model in step S320.

For example, the contact determination device may divide the candidate image into patches. Furthermore, the contact determination device may determine whether each of the patches is a contact occurrence patch by using the first artificial intelligence model. The contact determination device may determine the candidate image to be a valid image based on the number of contact occurrence patches included in the candidate image.

For example, the contact determination device may determine the candidate image to be a valid image when the number of contact occurrence patches included in the candidate image exceeds the first reference number or when the number of consecutively arranged contact occurrence patches included in the candidate image exceeds the second reference number. The content of these steps may be similar to steps S260 and S270 of FIG. 5.

For example, the contact determination device may input a candidate image to the second artificial intelligence model and acquire a probability value corresponding to each class. The contact determination device may determine a first frame adapted to output a value within a preset range of, e.g., 0.7 to 1.3 to be a valid image.

For example, the contact determination device may input a candidate image to the second artificial intelligence model and acquire a contact pressure value from the second artificial intelligence model. The contact determination device may determine that contact has occurred when the pressure value exceeds a preset reference value. Furthermore, the contact determination device may determine the candidate image to be a valid image when the pressure value falls within a preset range. In this case, the contact determination device may determine both whether there is contact and the degree of contact pressure by using the second artificial intelligence model.

For example, the contact determination device may determine the time when the valid image is acquired to be the time when the contact pressure between the ultrasound probe and the body part is in an appropriate state. Although the first and second artificial intelligence models are described as separate components herein, they may be implemented as a single integrated model.

The contact determination device may successively acquire candidate images from the ultrasound probe and successively perform step S320. When the contact determination device determines a plurality of valid images, the contact determination device may determine the time when a first valid image is determined to be the time when the contact pressure between the ultrasound probe and the body part is in an appropriate state.

The above description of the present disclosure is intended for illustrative purposes, and those having ordinary skill in the art to which the present disclosure pertains will understand that it may be easily modified into other specific forms without changing the technical spirit or essential features of the present disclosure. Therefore, it should be understood that the embodiments described above are exemplary and not limiting in all respects. For example, each component described as a single form may be implemented in a distributed form, and likewise, components described as distributed may be implemented in a combined form.

The scope of the present disclosure is defined by the claims described below rather than the detailed description above, and all changes or modifications derived from the meanings and scope of the claims and their equivalent concepts should be interpreted as being included in the scope of the present disclosure.

1. A method of determining contact between an ultrasound probe and a body part, the method being performed by a computing device including at least one processor, the method comprising:
acquiring a plurality of reference images via the ultrasound probe;
generating a contact determination criterion for determination of whether there is contact between the ultrasound probe and the body part and a valid pressure range for contact pressure based on the plurality of reference images;
acquiring a candidate image via the ultrasound probe;
determining whether the candidate image satisfies the contact determination criterion and the valid pressure range; and
when the candidate image satisfies the contact determination criterion and the valid pressure range, determining the candidate image to be a valid image acquired in a state where a contact pressure between the ultrasound probe and the body part is appropriate.

2. The method of claim 1, wherein the generating comprises:
generating the contact determination criterion so that, when a similarity between the plurality of reference images and the candidate image exceeds the contact determination criterion, it is determined that contact has occurred between the ultrasound probe and the body part.

3. The method of claim 2, wherein the generating the contact determination criterion comprises:
calculating similarities between the plurality of reference images, and generating the contact determination criterion based on a smallest value of the calculated similarities.

4. The method of claim 3, wherein the generating the contact determination criterion comprises:
dividing each of the plurality of reference images into patches; and
generating the contact determination criterion based on a smallest value of similarities between patches generated from the plurality of reference images.

5. The method of claim 2, wherein the determining whether the candidate image satisfies the contact determination criterion and the valid pressure range comprises:
when the similarity between a patch of the plurality of reference images and a patch of the candidate image satisfies the contact determination criterion, determining the patch of the candidate image to be a contact occurrence patch.

6. The method of claim 5, wherein the determining the candidate image to be a valid image comprises:
when a number of contact occurrence patches included in the candidate image exceeds a first reference number included in the valid pressure range, determining the candidate image to be the valid image.

7. The method of claim 5, wherein the determining the candidate image to be a valid image comprises:
when a number of consecutively arranged contact occurrence patches included in the candidate image exceeds a second reference number included in the valid pressure, determining the candidate image to be the valid image.

8. The method of claim 1, further comprising:
determining a time when the valid image is acquired to be a time when the contact pressure between the ultrasound probe and the body part is in an appropriate state.

9. A method of determining contact between an ultrasound probe and a body part, the method being performed by a computing device including at least one processor, the method comprising:
acquiring a candidate image via the ultrasound probe; and
determining whether the candidate image is a valid image acquired in a state where a contact pressure between the ultrasound probe and the body part is appropriate by using a first artificial intelligence model trained to determine whether there is contact between the ultrasound probe and the body part or a second artificial intelligence model trained to infer a contact pressure between the ultrasound probe and the body part.

10. The method of claim 9, wherein the determining comprises:
dividing the candidate image into patches;
determining whether each of the patches is a contact occurrence patch by using the first artificial intelligence model; and
determining the candidate image to be the valid image based on a number of contact occurrence patches included in the candidate image.

11. The method of claim 10, wherein the determining comprises:
when the number of contact occurrence patches included in the candidate image exceeds a first reference number, determining the candidate image to be the valid image.

12. The method of claim 10, wherein the determining comprises:
when the number of consecutively arranged contact occurrence patches included in the candidate image exceeds a second reference number, determining the candidate image to be the valid image.

13. A computer program stored in a computer-readable storage medium, the computer program, when executed on at least one processor, causing the processor to perform operations for determining contact between an ultrasound probe and a body part, wherein the operations comprise:
acquiring a plurality of reference images via the ultrasound probe;
generating a contact determination criterion for determination of whether there is contact between the ultrasound probe and the body part and a valid pressure range for contact pressure based on the plurality of reference images;
acquiring a candidate image via the ultrasound probe;
determining whether the candidate image satisfies the contact determination criterion and the valid pressure range; and
when the candidate image satisfies the contact determination criterion and the valid pressure range, determining the candidate image to be a valid image acquired in a state where a contact pressure between the ultrasound probe and the body part is appropriate.
